(19) United States
(12) Patent Application Publication
(10) Pub. No.: US 2011/0002966 A1
(43) Pub. Date: Jan. 6, 2011

(54) Vaginal Suppository System and Method

(76) Inventors: Alex Lovett, Southhampton, NY (US); Marge Siebert, New York, NY (US)

Correspondence Address: JAY M. SCHLOFF, 6960 Orchard Lake Road, Suite 315, West Bloomfield, MI, 48322, US

(21) Appl. No.: 12/822,804

(22) Filed: Jun. 24, 2010

Related U.S. Application Data
(60) Provisional application No. 61/222,277, filed on Jul. 1, 2009.

(51) Int. Cl. A61K 9/00; A61K 36/49; A61K 36/00; A61K 36/752; A61K 36/736; A61K 33/30; B28B 1/14; A61P 15/00

(52) U.S. Cl. 424/400; 424/771; 424/773; 424/736; 424/735; 424/725; 424/641; 264/299

(57) ABSTRACT

A vaginal suppository system contains a formulation comprising active and inactive ingredients, which active ingredients may be nutraceuticals, herbs, vitamins, minerals and other bioactive agents. In an embodiment, the active ingredients comprise Oak gall, true unicorn root, bitter orange peel, tropical almond, heartsease, and zinc oxide. The formulation may be contained in a soluble encapsulation or a suspension for administration of the formulation in a vagina. A method of preparing a vaginal suppository system and a method of treating a vagina with a formulation comprising active and inactive ingredients are also disclosed.

### CROSS-REFERENCE TO RELATED APPLICATIONS

The present application claims priority under 35 U.S.C. §119 on the U.S. Provisional Patent Application Ser. No. 61/222,277, filed on Jul. 1, 2009, the disclosure of which is incorporated by reference.

### FIELD OF THE DISCLOSURE

The present disclosure relates to a vaginal suppository system and method for administering a formulation including nutraceuticals, herbs, vitamins, minerals and other ingredients, to promote vaginal tightening, vaginal lubrication, microbial content in the vagina, vaginal rejuvenation and sexual response/function.

### BACKGROUND

Sexual response in both men and women results from a complex interplay of psychological, hormonal, and other physiological influences. Sexual response during intercourse may be disrupted due to problems attendant with female sexual dysfunction. Examples of problems related to female sexual dysfunction include, but are not limited to, a loosened, relaxed, stretched, prolapsed or atrophied state of the vagina; loss of sexual arousal; pain or discomfort during intercourse (dyspareunia); diminished blood flow to the vagina and genital area; inability to achieve orgasm; and general sexual dissatisfaction.

Enumerable factors that contribute to the foregoing sexual dysfunction include, but are not limited to, physiological problems and changes, hormonal imbalances, medications (such as antidepressants), vaginal infections, vascular conditions, pregnancy and childbirth, aging, and gravity.

A loosened state of the vagina can contribute to female sexual dysfunction, as natural swelling and lubrication of the vagina that occurs during an arousal phase of an intercourse may take longer to occur in the loosened state. Further, loosening of the vagina prevents, circumvents, and/or compromises friction against the penis during intercourse, which friction is a major component of sexual sensation and gratification for both the male and female partner. The compromised friction may also reduce sensitivity of the clitoris, a sexual organ present above the vagina of the woman, which organ functions to induce sexual response/function. As a result, intercourse may be difficult and/or frustrating. Further, beyond the loss of sexual satisfaction, the woman may develop a feeling of frustration, a loss of self-esteem and anxiety about her relationship with her partner.

To address these above-described problems related to female sexual dysfunction, various creams have been developed for application in the vagina. These creams purportedly tighten the canal of the vagina (hereinafter referred to as “vaginal canal”). Although the readily available and conventional creams include effective ingredients, manual application of such creams is not easily accomplished to deliver a useful quantity in the vagina. More specifically, the conventional creams are incapable of introducing the effective ingredients directly inside the entire length of the vaginal canal. Usually, a significant portion of the quantity applied is likely to remain external to the vaginal canal (such a portion is hereinafter referred to as “residual cream”), reducing the effectiveness of a cream. Further, such residual cream may be undesirably left on hands, underwear, thighs and buttocks of the user; on bedding; and on or in other undesired places.

Furthermore, conventional creams carry a potential contamination risk as microbes and debris may easily be introduced (by the fingers of a user) into jars, tubes, bottles and caps that enclose and store these conventional creams. In addition, the conventional creams may include hydrophobic and oil-based excipients, which may dissolve latex-based condoms. Accordingly, use of such conventional creams may increase the risk of sexually transmitted diseases and pregnancy during intercourse.

Alternatively, various vaginal procedures, such as vaginal tightening rejuvenation surgery, exist for the treatment of the loosened state and other such abnormalities of the vagina that are responsible for causing loss of sexual response in women. Vaginal tightening rejuvenation surgery enables tightening of vaginal canal of a woman for enhancing sexual response for both the woman and her partner during an intercourse. However, such surgery also implicates pain, risk and a high cost. Further, due to safety concerns, such vaginal procedures have become quite unpopular. (See, for example, Committee Opinion #378 of the American College of Obstetricians and Gynecologists, entitled “Vaginal Rejuvenation and Cosmetic Vaginal Procedures,” published in the September 2007 issue of Obstetrics & Gynecology.)

Accordingly, there is a need for effectively increasing female sexual response and preventing female sexual dysfunction to create more satisfactory sexual experiences for a woman and her partner. Further, there is a need for providing moisturizing, toning and replenishing effects for vaginal rejuvenation and vaginal lubrication in women, in an effective and a hygienic manner. In addition, there is a need for providing hormonal support to the vagina and for preventing vaginal prolapse. Moreover, there is a need for providing antimicrobial effects for preventing and controlling microbial infections in the vagina.

### SUMMARY OF THE DISCLOSURE

Therefore, it is an object of the present disclosure to obviate the above and other disadvantages from the existing art by providing a method and a system for promoting vaginal tightening to create more satisfactory sexual experiences for women and their partners.

It is another object of the present disclosure to provide a method and a system for moisturizing, toning and replenishing a vagina for vaginal rejuvenation and vaginal lubrication, in an effective and a hygienic manner.

It is yet another object of the present disclosure to provide a method and a system for providing hormonal support to a vagina and for preventing vaginal prolapse.

It is still another object of the present disclosure to provide a method and a system for preventing and controlling microbial infections in the vagina.

These together with other aspects of the present disclosure, along with the various features of novelty that characterize the disclosure, are pointed out with particularity in the claims annexed hereto and form a part of this disclosure.

### DETAILED DESCRIPTION OF THE DISCLOSURE

The exemplary embodiments described herein detail for illustrative purposes are subject to many variations in structure and design. It should be emphasized, however, that the present disclosure is not limited to a vaginal suppository system, as shown and described. It is understood that various omissions and substitutions of equivalents are contemplated as circumstances may suggest or render expedient, but these are intended to cover the application or implementation without departing from the spirit or scope of the claims of the present disclosure. The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

As used herein, the clause “suppository system” refers to a drug/pharmaceutical/nutraceutical/aesthetic/herbal formulation delivery system that is applied or inserted into vagina of a woman (to serve as a vaginal suppository system). More specifically, the suppository system dissolves inside the vagina to deliver the formulation. The suppository system may be used to deliver both a locally-acting and a systemically-acting formulation.

As used herein, the clause “vaginal suppository formulation” refers to a pharmaceutical/nutraceutical/aesthetic/herbal preparation having specific ingredients, and more specifically, to a solution, a cream, a lotion, a gel, an emulsion, a cream gel, an ointment, or any other suitable topical dosage form, or any other suitable locally acting form of the pharmaceutical preparation. The vaginal suppository formulation may be either in a liquid form, a semisolid form, or a solid form. Further, the vaginal suppository formulation is devised to form a vaginal suppository system for application in the vagina of a female.

As used herein, the term “antimicrobial” refers to an effect of ingredients, which ingredients are capable of killing or thwarting the growth of various pathogens/microbes such as bacteria, viruses, fungi and prions.

As used herein, the term “active ingredient” refers to nutraceuticals, herbs, vitamins, minerals and such other bioactive agents, which are capable of treating an abnormal, distressed, or sexually insensitive state (such as a loosened state) of vagina of a woman for enhancing sexual response/function thereof.

As used herein, the term “excipient” refers to an inactive ingredient such as a carrier/vehicle for the active ingredients of a pharmaceutical formulation. More specifically, when the active ingredients are not easily administered to and absorbed by a human body, an excipient may be used for dissolving or mixing with the active ingredients. Further, an excipient may be used to bulk up formulations with highly potent active ingredients, to allow administration of a convenient and accurate dosage of the formulations. For a vaginal suppository system, an excipient forms a suppository base for delivering the active ingredients of a formulation of a pharmaceutical/nutraceutical/aesthetic/herbal preparation within the vagina of a woman.

As used herein, the term “synergistic” refers to the additive effects of different active ingredients and/or excipients when combined with each other.

In one aspect, the present disclosure provides a vaginal suppository system for application/insertion in vagina of a woman. The vaginal suppository system is prepared using a formulation (hereinafter referred to as “vaginal suppository formulation”). The vaginal suppository formulation includes at least one active ingredient (hereinafter interchangeably referred to as “active ingredients”). The active ingredients may either be nutraceuticals, herbs, vitamins, minerals and other bioactive agents. Suitable examples of the active ingredients include, but are not limited to, Oak gall, True unicorn root, Bitter orange peel, Tropical almond, Heartsease, Curcuma (turmeric), Pueraria mirifica, Gambir, Parameria barbata, Tongkat ali, Ginko biloba, Labisia pumilia, Muira puama, L-arginine, dimethylaminoethanol (DMAE), vitamin E and zinc oxide.

In a preferred embodiment of the present disclosure, Oak gall, also known as Quercus infectoria, is employed as the at least one active ingredient of the vaginal suppository formulation. In general, galls may be found on many species of Oak (such as Quercus spp.), and are produced when a gall wasp deposits eggs thereof in a bud of an Oak leaf, thereby infecting the Oak leaf. As the Oak leaf and wasp larvae develop, the infected Oak leaf reacts by producing gall tissue. The gall tissue surrounds the wasp larvae, which emerge from Oak gall upon maturity to complete life cycle thereof. Oak gall includes high levels (about 60 to 70 percent by weight of the total weight) of tannins (gallotannins); and phenol carboxylic acids (gallic acid at about 3 percent by weight of the total weight and ellagic acid at about 2 percent by weight of the total weight). Both the tannins and the phenol carboxylic acids provide a constrictive action. Accordingly, Oak gall is capable of causing constriction of tissues along vaginal walls and vaginal mucosal surfaces of the woman, thereby causing vaginal tightening. Further, Oak gall aids in preventing and controlling microbial infection in mucous membranes, skin and other parts of the vagina of the woman. Oak gall may be prepared in cut/sliced and dried form; ground form; powdered form; whole form; and tinctured form. For preparing the tinctured form, Oak gall may be powdered and subsequently mixed with spirit of wine in a ratio of about 1:5.

Further information about the properties of Oak gall may be found in Soon L K, Hasni E (2005) Consumption of traditional medicines/herbs postpartum among the Kelantanese Malay women. Paper presented at the 10th. National Conference of Medical Sciences (NCMS), School of Medical Sciences, Universiti Sains Malaysia on 22 May 2005; and in Soon L K, Hasni E, Law K S, Waliullah S S, Farid C G, and Syed Mohsin S. 2007. Ultrastructural findings and elemental analysis of Quercus infectoria Oliv. Annals of Microscopy 7:32-37.

True unicorn root (Colicroot), also known as Aletris farinosa, is an herb that may be employed as the at least one active ingredient of the vaginal suppository formulation for maintaining structure of the vagina of the woman. True unicorn root is also capable of providing hormonal support to the vagina; and treating vaginal prolapse (restructuring of vagina following the vaginal prolapse), amenorrhea, and dysmenorrhea. True unicorn root is a member of the lily family that produces a spike of white flowers. Further, True unicorn root includes volatile oils, resins, starches and saponins. True unicorn root may be collected, dried and ground into a powder form for the preparation of the vaginal suppository formulation. Dosage for the powdered form may vary from about 0.3 to about 0.6 grams (g) for three times per day.

Further information about true unicorn root is available in Gruenwald J, Brendler T, Jaenicke C., eds. 2007. Physicians Desk Reference for Herbal Medicines. Fourth Edition. Thomson Healthcare Inc.

Peel of Bitter orange (hereinafter interchangeably referred to as “Bitter orange peel”), which is also known as Citrus aurantium, may be employed as the at least one active ingredient of the vaginal suppository formulation for moisturizing, toning and replenishing the vagina and walls thereof; and for maintenance of the vaginal structure. Bitter orange peel may also be capable of providing hormonal support to the vagina; and treating vaginal prolapse (restructuring of vagina following a vaginal prolapse). Bitter orange peel includes volatile oils (such as limonene, nerol, geraniol, linalool, linalyl acetate, neryl acetate, geranyl acetate, citronellyl acetate, methyl anthranilate); flavonoids (neohesperidin dyhydrochalcone, naringin, sinensetin, nobiletin, tangeretin); and furocoumarins.

Bitter orange peel is an outermost layer of dried peel from ripe fruits of a bitter orange plant. Further, for the preparation of the vaginal suppository formulation, Bitter orange peel may be dried and the outermost layer may then be removed from an underlying white pulp layer. The outermost layer may then be powdered, ground, or cut as desired. It should be understood that Bitter orange peel may be obtained from various varieties (such as Seville orange and Bergamot orange) of Bitter orange plant. Daily dosage of Bitter orange peel may vary from about 4 to about 6 g; or about 2 to about 3 g for tincture form of Bitter orange peel obtained after mixing Bitter orange peel with spirit of wine in a ratio of about 1:5.

Further information about bitter orange peel is available in Gruenwald J, Brendler T, Jaenicke C., eds. 2007. Physicians Desk Reference for Herbal Medicines. Fourth Edition. Thomson Healthcare Inc.

Tropical almond, also known as Terminalia chebula may be employed as the at least one active ingredient of the vaginal suppository formulation to counter aging/disease, skin changes, undesired discharges; maintaining vaginal structure; and to nourish vaginal tissues. Tropical almond may also be capable of providing hormonal support to the vagina. Tropical almond includes tannins (gallotannins including terchebulin, terflavin A, punicalagin, corilagin, chebulic acid, chebulinic acid) at an amount of about 20 to 45 percent by weight of the total weight; monosaccharides and oligosaccharides (including D-glucose; D-fructose; saccharose) at an amount of about 9 percent by weight of the total weight; fruit acids (quinic acid; shikimic acid); and fatty oils. For the preparation of the vaginal suppository formulation, Tropical almond may be ground finely prior to use. Further, maximum daily dosage of about 1000 milligrams (mg) is sufficiently effective.

Further information about tropical almond is available in Gruenwald J, Brendler T, Jaenicke C., eds. 2007. Physicians Desk Reference for Herbal Medicines. Fourth Edition. Thomson Healthcare Inc.

Heartsease, also known as Viola tricolor, is a wildflower (having herbal properties) that may be employed as the at least one active ingredient of the vaginal suppository formulation as a salve/soothing agent for treating skin disorders/ailment; for skin toning; for providing hormonal support; and for moisturizing skin/mucous membranes of the vagina. Heartsease includes a small amount (about 0.2 to about 0.4 percent by weight of the total weight) of flavonoids including ruitin, scoparin, saponarine, and violanthin; phenol carboxylic acids (salicylic acid, violutoside); mucilage (about 10 percent by weight of the total weight); tannins (about 2 to about 5 percent by weight of the total weight); umbelliferone; and optionally triterpanes. Further, a powdered form or a cut form of Heartsease may be employed in the vaginal suppository formulation of the present disclosure.

Further information about heartsease is available in Gruenwald J, Brendler T, Jaenicke C., eds. 2007. Physicians Desk Reference for Herbal Medicines. Fourth Edition. Thomson Healthcare Inc.

Zinc oxide is an inorganic compound that may be employed as the at least one active ingredient of the vaginal suppository formulation as bioadhesive agent for increasing the adhesive properties of the suppository formulation.

The vaginal suppository formulation further includes at least one excipient (hereinafter interchangeably referred to as “excipients”) capable of carrying/supporting the active ingredients. Suitable examples of the excipients include, but are not limited to, polyethylene glycol, cocoa butter, lecithin and shea butter. The excipients allow melting or dissolution of the vaginal suppository system, prepared using the excipients as a suppository base, at a temperature of about 37 degrees Celsius (i.e. human body temperature) for release of the active ingredients in the vagina of the user. Further, the excipients provide consistency to the vaginal suppository formulation of the present disclosure.

In one embodiment of the present disclosure, the vaginal suppository formulation includes polyethylene glycol as the at least one excipient. Polyethylene glycol is a polymer with the following structural formula:

[H(OCH2CH2)ₙOH]

Polyethylene glycol acts as a water soluble excipient for the vaginal suppository formulation. Further, polyethylene glycol is readily soluble in aromatic hydrocarbons, and slightly soluble in aliphatic hydrocarbons. Furthermore, polyethylene glycol may function as a liquid or solid matrix. Moreover, polyethylene glycol may function as an emulsifier. Additionally, polyethylene glycol resists microbial/mold growth, and is incapable of undergoing hydrolysis or deterioration during storage. Accordingly, polyethylene glycol serves as an effective excipient for the vaginal suppository formulation of the present disclosure.

Although polyethylene glycol is known to degrade plastics, polyethylene glycol is incapable of degrading latex. Accordingly, polyethylene glycol is a suitable excipient for delivery of the active ingredients of the vaginal suppository formulation applied in the form of the vaginal suppository system prior to a sexual intercourse. More specifically, use of polyethylene glycol as a hydroscopic excipient in the vaginal suppository formulation avoids dissolution of latex condoms, thereby avoiding any risk of sexually transmitted diseases and pregnancy during the sexual intercourse.

Polyethylene glycol may be procured in a wide range of molecular weights and viscosities to provide a desired melting point for use in the vaginal suppository formulation of the present disclosure. Melting point of polyethylene glycol varies significantly depending upon molecular weight thereof, as shown in Table 1, which provides melting point values for polyethylene glycol having varying polymer chain lengths.

TABLE 1

| Average Molecular | Molecular Weight Range | Melting Point |
|---|---|---|
| Weight (Daltons) | (Daltons) | (Degrees Celsius) |
| 200 | 190-210 | — |
| 400 | 380-420 | 4-8 |
| 600 | 570-630 | 20-25 |
| 1500 | 1300-1600 | 44-48 |
| 4000 | 3000-4700 | 54-58 |

Polyethylene glycol of a suitable chain length or a mixture of different polyethylene glycols may be employed as the at least one excipient such that the vaginal suppository system prepared using the at least one excipient melts at the human body temperature. Particularly, polyethylene glycol having an average molecular weight of about 1000 Daltons is capable of providing a desired melting point and consistency for preparing an effective vaginal suppository system. Further, such vaginal suppository system upon melting (while being inserted in the vagina and/or in contact with various vaginal secretions), enables a homogenous and consistent release of the vaginal suppository formulation, and more specifically, of the active ingredients, within the vagina with the help of polyethylene glycol.

The vaginal suppository formulation may optionally include an additional emulsifier in conjunction with polyethylene glycol. A suitable example of the emulsifier is lecithin. More specifically, sunflower-based lecithin may be employed in the vaginal suppository formulation of the present disclosure. As mentioned above, lecithin may also be used as the at least one excipient in the presence or absence of polyethylene glycol, in the vaginal suppository formulation.

Lecithin may be isolated from many plant sources, and may thereby be obtained as an all-natural (non-synthesized) ingredient, or as a modified ingredient. Lecithin is a natural emulsifier, surfactant, and lubricant that may be metabolized by humans. Further, lecithin allows the volatile oils of Bitter orange peel and the fatty oils of Tropical almond to be completely incorporated or homogenized with other ingredients of the vaginal suppository formulation. Furthermore, as a lubricant, lecithin supports the soothing/salve properties of Heartsease. Lecithin may also provide firmness to cocoa butter, which may be used as the at least one excipient in the absence or presence of polyethylene glycol in the vaginal suppository formulation.

In another embodiment, the suppository formulation may be delivered as a suspension, encapsulated in a water-soluble soft capsule designed and sized appropriately for vaginal delivery. Encapsulation materials may include, but are not limited to, gelatin (i.e. softgel), carrageenan, starch, polyvinyl alcohol, and the like.

Based on the foregoing, it may be concluded that the active ingredients of the vaginal suppository formulation of the present disclosure have a variety of properties for treating or preventing various abnormal, distressed, or sexually insensitive states of the vagina. Such independent properties therefore serve as sexual enhancement properties. Further, the active ingredients may also work in a synergistic manner to promote sexual response/function, by causing vaginal tightening, for creating more satisfactory sexual experiences for both the user and her partner.

In another aspect, the present disclosure provides a method for preparing the vaginal suppository system from the above-described vaginal suppository formulation. The method includes heating the at least one excipient. The at least one excipient may be liquefied at a lowest possible temperature in order to attain a form, which is capable of preserving activity of the active ingredients of the vaginal suppository formulation. Further, the method includes selecting and then mixing the at least one active ingredient with the heated form of the at least one excipient. Furthermore, the method includes inserting the mixture into a suitable suppository mold. More specifically, the mixture may be cooled and injected into a plastic suppository strip mold. Subsequently, the mixture setting within the suppository mold may then be cooled to an ambient temperature (room temperature) to cast/form the vaginal suppository system. More specifically, casting of the mixture into the vaginal suppository system may be achieved using an egg-shaped mold, a globular mold, an oviform mold or a cone-shaped mold. Alternately, the mixture of the heated form of the at least one excipient and the at least one active ingredient may manually be formed into a vaginal suppository system of a desired shape, such that, the vaginal suppository system is capable of being inserted/applied within the vagina in an effective manner. The vaginal suppository system may be prepared in a semisolid form, or a solid form, or a gel-like form.

In an embodiment, the method includes heating polyethylene glycol to approximately 120 to 160 degrees Fahrenheit, and then cooling said polyethylene glycol to approximately 110 to 150 degrees Fahrenheit. The method further includes combing at least one active ingredient of the vaginal suppository formulation (in ground powdered form, for example), mixing the ingredient(s) with the polyethylene glycol, and pouring the resultant mixture into a mold. The mold may be an aluminum cast mold, a plastic shell mold, or a flexible or hard rubber mold, for example. The method further includes cooling the mixture until the mixture congeals. In another embodiment, the method may include encapsulating the congealed mixture.

Subsequent to either casting/formation or prior to use, the vaginal suppository system may be removed/dispensed from the suppository mold. In an embodiment of the present disclosure, the vaginal suppository system may be formed in a dispensing cup fitted with the suppository mold. Further, an accompanying tool may be provided to push the vaginal suppository system out through a bottom portion of the suppository mold or the dispensing cup. The tool, as employed herein, may be a tool known in the art for removing/dispensing existing suppositories from a mold, such as, but not limited to, a hammer, a mallet, a spoon, and the like.

In yet another aspect, the present disclosure provides a method for treatment of vagina of a woman. The term, “treatment” refers to promoting vaginal tightening to create more satisfactory sexual experiences for both men and women, moisturizing; toning and replenishing vagina of women for vaginal rejuvenation and vaginal lubrication, in an effective and a hygienic manner; providing hormonal support to the vagina for preventing vaginal prolapse; and preventing and controlling microbial infections in the vagina.

The method includes administration of the above-described vaginal suppository system into the vagina of the woman by a direct application or insertion in the vagina. The vaginal suppository system may be applied in a manner such that the active ingredients are directly and/or topically introduced inside the entire length of vaginal canal. In one embodiment, the vaginal suppository system is inserted as a semi-solid form, which dissolves inside the vagina to deliver the active ingredients within the vagina. The vaginal suppository system of the present disclosure therefore provides optimized placement of the active ingredients in the vagina for a proper effect of the active ingredients. Alternatively, the vaginal suppository system may be inserted as a solid or a gel-like form into the vagina.

Further, the vaginal suppository system may only be used once. Such a single use prevents any occurrence of contamination of the vaginal suppository formulation and the active ingredients thereof, and thus prevents contamination of the vagina in which the system is inserted. Accordingly, the use of the vaginal suppository system promotes vaginal tightening, vaginal rejuvenation and vaginal lubrication, in an effective and a hygienic manner.

In an embodiment of the present disclosure, the vaginal suppository system may be inserted in the vagina of a woman at a time period of about 10 to about 30 minutes prior to sexual intercourse. In another embodiment of the present disclosure, the vaginal suppository system may be inserted in the vagina of a woman at a time period of about 12 hours prior to sexual intercourse. Application of the vaginal suppository system may be repeated as often as necessary or desirable for an enhanced sexual response/function. Further, daily use may help promote sustained vaginal rejuvenation.

A vaginal suppository formulation for the vaginal suppository system of the present disclosure is explained in detail in conjunction with the following non-limiting example. However, one of ordinary skill in the art, and based on a reading of this detailed description, would recognize that, the specific example is intended to illustrate, and not limit, the scope of the present disclosure.

### EXAMPLE

Composition of an exemplary vaginal suppository formulation (having a total mass of 15 g) of the present disclosure, is provided in table 2. The exemplary vaginal suppository formulation is capable of promoting vaginal tightening, vaginal rejuvenation and vaginal lubrication, and in providing hormonal support to a female's vagina. Further, the exemplary vaginal suppository formulation has antimicrobial effects. The exemplary vaginal suppository formulation may include polyethylene glycol with an average molecular weight less than or equal to about 1000 Daltons to provide a desired melting point and consistency for the exemplary vaginal suppository formulation.

TABLE 2

| | | Percent by | Percent by | Maximum |
|---|---|---|---|---|
| Common | | Weight of Active | Weight of Total | Daily Dose |
| Name | Use | Ingredients (%) | Ingredients (%) | (mg)* |
| Oak gall | Active | 40 | 2.6 | — |
| | ingredient | | | |
| True unicorn | Active | 25 | 1.7 | — |
| root | ingredient | | | |
| Bitter orange | Active | 10 | 0.7 | — |
| (peel) | ingredient | | | |
| Tropical | Active | 10 | 0.7 | 1000 |
| almond | ingredient | | | |
| Heartsease | Active | 15 | 1.0 | — |
| | ingredient | | | |
| Polyethylene | Excipient | — | 52.8 | 2 g/kg of bodyweight |
| glycol 400 | | | | (for aggregate of |
| | | | | PEG 400 and 4000) |
| Polyethylene | Excipient | | 30.9 | See box directly |
| glycol 4000 | | | | above |
| Zinc Oxide | Active | | 9.5 | — |
| | Ingredient, | | | |
| | Colorant | | | |
| Total | — | 100 | 100 | — |

*maximum daily dose for Polyethylene Glycol is given in grams per kilogram of bodyweight

In another embodiment, the exemplary vaginal suppository formulation includes lecithin as an additional excipient and as an emulsifier. It should be understood that the total amount of excipients employed in the exemplary vaginal suppository formulation may be increased or decreased to achieve a specific consistency, as desired.

It should be apparent to a person skilled in the art that depending on a specific size and weight (as desired) of the vaginal suppository system, it may be useful or necessary to increase or decrease amount (in percent by weight) of each of the active ingredients, each of the excipients and/or the emulsifier in the vaginal suppository formulation of the present disclosure.

Based on the foregoing, the present disclosure provides a vaginal suppository system to deliver nutracueticals, herbs, vitamins, minerals and bioactive agents, which individually and/or synergistically contribute to a heightened sexual response by promoting vaginal tightening, vaginal rejuvenation, vaginal lubrication and microbial control in the female vagina. The vaginal tightening, vaginal rejuvenation, vaginal lubrication and antimicrobial effects dramatically enhance sexual response/function (in the form of a sexual sensation) for both a woman and her partner during a sexual intercourse, resulting in a mutual sexual satisfaction.

The suppository system and methods disclosed herein provides for effective delivery of a both a locally-acting and a systemically-acting formulation directly inside the entire length of the vaginal canal. This delivery may be without potential contamination risk arising from microbes and debris that are associated with conventional creams.

The foregoing descriptions of specific embodiments of the present disclosure have been presented for purposes of illustration and description. They are not intended to be exhaustive or to limit the disclosure to the precise forms disclosed, and obviously many modifications and variations are possible in light of the above teaching. The exemplary embodiments were chosen and described in order to best explain the principles of the disclosure and its practical application, to thereby enable others skilled in the art to best utilize the disclosure and various other embodiments with various modifications as are suited to the particular use contemplated.

What is claimed is:

1. A vaginal suppository system, the system comprising a formulation, said formulation comprising at least one active ingredient, and at least one excipient.

2. The vaginal suppository system of claim 1, wherein the at least one active ingredient is Oak gall.

3. The vaginal suppository system of claim 1, wherein the at least one active ingredient is true unicorn root.

4. The vaginal suppository system of claim 1, wherein the at least one active ingredient is bitter orange peel.

5. The vaginal suppository system of claim 1, wherein the at least one active ingredient is tropical almond.

6. The vaginal suppository system of claim 1, wherein the at least one active ingredient is heartsease.

7. The vaginal suppository system of claim 1, wherein the at least one excipient is polyethylene glycol.

8. The vaginal suppository system of claim 1, wherein the at least one excipient is an emulsifier.

9. The vaginal suppository system of claim 1, wherein said formulation is contained in one of a suspension and an encapsulation.

10. The vaginal suppository system of claim 1, wherein said active ingredients of said formulation comprise Oak gall, true unicorn root, bitter orange peel, tropical almond, heartsease, and zinc oxide.

11. The vaginal suppository system of claim 10, wherein said excipient comprises polyethylene glycol 400 and polyethylene glycol 4000.

12. The vaginal suppository system of claim 10, wherein said excipient comprises polyethylene glycol 400, polyethylene glycol 4000, and an emulsifier.

13. A method for preparing a vaginal suppository system, the method comprising:
heating at least one excipient into a liquefied state,
mixing at least one active ingredient with the heated form of the at least one excipient,
inserting the mixed at least one excipient and at least one active ingredient into a suppository mold,
cooling the mixed at least one excipient and at least one active ingredient after their insertion into a suppository mold,
casting the cooled at least one excipient and at least one active ingredient within the suppository mold to form the suppository.

14. The method of claim 13, wherein the at least one active ingredient is ground into a powder before it is mixed with the heated form of the at least one excipient.

15. The method of claim 13, wherein the at least one excipient comprises polyethylene glycol 400 and polyethylene glycol 4000.

16. The method of claim 13, wherein said at least one active ingredient comprise Oak gall, true unicorn root, bitter orange peel, tropical almond, heartsease, and zinc oxide.

17. A method for treatment of a vagina, the method comprising:
administering a vaginal suppository system into the vagina,
said system comprising a formulation comprising at least one excipient and at least one active ingredient.

18. The method of claim 17, wherein said at least one excipient comprises polyethylene glycol 400 and polyethylene glycol 4000 and wherein said at least one active ingredient comprise Oak gall, true unicorn root, bitter orange peel, tropical almond, heartsease, and zinc oxide.

19. The method of claim 17, wherein said administering of said vaginal suppository system occurs 10 to 30 minutes before sexual intercourse.

20. The method of claim 17, wherein said administering of said vaginal suppository system occurs 12 hours before sexual intercourse.
